(19) United States
(12) Patent Application Publication
(10) Pub. No.: US 2009/0071368 A1
(43) Pub. Date: Mar. 19, 2009

(54) Process for the dispersion of fine-particle inorganic powders in liquid media, with use of reactive siloxanes

(75) Inventors: Klaus Steingrover, Saarbrucken (DE); Frank Tabellion, Saarbrucken (DE); Steffen Pilotek, Saarbrucken (DE); Michael Khim, Saarbrucken (DE)

Correspondence Address: BACHMAN & LAPOINTE, P.C., 900 CHAPEL STREET, SUITE 1201, NEW HAVEN, CT, 06510, US

(73) Assignee: Buhler PARTEC GmbH, Saarbrucken (DE)

(21) Appl. No.: 12/211,989

(22) Filed: Sep. 17, 2008

(30) Foreign Application Priority Data
Sep. 17, 2007 (DE) 10 2007 044 302.3

(51) Int. Cl. C09D 5/00; A61K 6/00

(52) U.S. Cl. 106/35; 106/481; 106/490; 524/261; 106/38.2

(57) ABSTRACT

The invention relates to a process for the dispersion of fine-particle surface-modified inorganic powders in liquid media, with use of siloxanes. A process for the preparation of a dispersion of inorganic particles in a liquid medium is described, in which inorganic particles which have been surface-modified so that they have at least one organic group on the surface are mixed in a liquid medium with an organosiloxane, where at least one organic group of the organosiloxane corresponds to the at least one organic group on the surface of the inorganic particles.

### BACKGROUND OF THE INVENTION

The invention relates to a process for the dispersion of inorganic powders in liquid media.

The use of powders as fillers in lacquers, in films, in coatings and in moulding compositions can improve a wide variety of properties, such as tensile strength and compressive strength, abrasion resistance, general mechanical stability, and processability. Functional fillers can moreover be used to introduce further properties into the materials, examples being colour through colour pigments, UV resistance, and magnetic, optical or electrical properties. The term pigments here is intended to comprise very generally by way of example fillers, colour pigments or functional pigments.

To ensure that the materials have homogeneous properties, it is essential to achieve excellent dispersion of the pigments in a liquid or viscous medium. This is relatively difficult to achieve when the particles of the pigments used are relatively fine and when the compatibility between pigment and medium becomes poorer. An important factor in this context is the viscosity and the stability of the mixture. Addition of fine-particle pigments usually increases viscosity. Viscosity can also rise unacceptably after the dispersion process.

There is therefore wide-ranging prior art for promoting the dispersion of pigments of liquid media, either by adding wetting or dispersing additives or by modifying the powder surface to improve dispersibility.

Wetting agents and dispersing agents are used to provide compatibility between powder and medium. By way of example, ionic, non-ionic, amphiphilic and polymeric compounds having different chemical structures have been used, these being respectively suitable for various dispersion processes. Ionic structures, for example, are mainly used for oxidic powders, while non-ionic surfactants are often used in the dispersion of non-oxidic powders. Combination of various structures in organic polymers is intended to achieve the widest possible application profile of dispersing agents with respect to the powders and dispersion media used.

DE-A-4236337 describes the use of polyacrylic esters as dispersing agents, these being obtained via transesterification of polyacrylates.

DE-A-10200416479 relates to the use of polyesters containing carboxylate groups, as dispersing agents for pigment concentrates for the colouring of thermoplastics. DE-A-10200444879 describes the use of copolymers as wetting agents and dispersing agents, these being obtainable via copolymerization of unsaturated monocarboxylic acid derivatives, of polyalkyleneoxy allyl ethers and, if appropriate, of further monomers.

DE-A-10232908 describes the use of specific polysiloxanes, containing phenyl derivatives, as dispersing agents for aqueous media. EP-A-546406 and EP-A-546407 relate to the use of organofunctional polysiloxanes having ester groups and having long-chain alkyl groups for the modification of fine particles, such as pigments or fillers, or of glass fibres, where the siloxanes can react by way of their organic functional groups with the reactive particle surface.

A general disadvantage with the use of dispersing additives is the increase in chemical complexity caused in essence by introducing a contaminant into the overall mixture. It is desirable to minimize the number of different components in the system.

Another method used to improve dispersibility of inorganic particles is modification of the particle surface, as found by way of example in the Degussa brochure “Sivento Silanes for Treatment of Fillers and Pigments”; R. Janda, Kunststoff-Verbundsysteme [Plastics Composite Systems], VCH Verlag 1990, p. 98; EP-A-753549; and W. Noll, Chemie und Technik der Silicone [Chemistry and Technology of Silicones], p. 524, Weinheim 1968.

It is also possible to use surface modification to functionalize inorganic powders. By way of example, functional organic groups can be anchored on the surface of the particles. The powders are surface-modified by treatment with modifiers which interact with the surface of the particles. The amount of the modifier to be used here is in essence determined by the surface area to be modified. From 1 to 10% by weight, based on the powder, are usually proposed (e.g. for silanes in the brochure “Sivento Silanes for Treatment of Fillers and Pigments”, Degussa AG, Frankfurt a.M.). The use of excess modifier, which does not interact with the surface, can make treatment of the material more difficult, and by way of example relatively volatile non-interacting modifiers can be removed concomitantly to some extent during removal of the solvent.

WO 93/21127 relates to a process for the preparation of surface-modified nanoscale ceramic powders, and it is stated here that modification of the surface is required in the case of extremely fine-particle nanoscale powders, in order to avoid agglomeration and to improve dispersibility.

DE-A-10304849 describes a chemo-mechanical preparation of functional colloids via combination of mechanical reactive comminutation and surface modification for the preparation of dispersions of fine particles.

The modification generally improves dispersibility, but, surprisingly, is not generally sufficient to achieve high filler levels of fine-particle inorganic powders in liquid media without a drastic viscosity increase.

WO 2004/24811 describes a process for the preparation of nanocomposites, by modifying agglomerated nanopowders in an organic solvent, e.g. using silanes. The powders thus modified are either further processed as dispersion or dried prior to their further processing. The process is restricted to the processing of agglomerated powders. When silanes are used, a hydrolysis-condensation reaction is carried out in the presence of the powders, thus permitting binding of the reactive silane species to the powder surface. The examples use relatively large amounts of silanes, leading to formation of nanocomposites. However, stable dispersions are not obtained, and this greatly increases the difficulty of subsequent further processing, e.g. via solvent exchange and further processing. The difficulty of further processing via drying and subsequent handling of the powders is moreover made markedly more difficult, in particular if the vapour pressure of the silane is comparatively high.

### SUMMARY OF THE INVENTION

The object of the present invention therefore consisted in providing a process which can prepare stable dispersions composed of fine-particle inorganic powders in high concentrations in liquid media, including viscous media, without any need to accept the disadvantages mentioned of the prior art.

A further object of the present invention consisted in providing a process which can disperse surface-modified and functionalized particles at high concentration.

Surprisingly, the object was achieved via a two-stage process in which the surface of the powder particles is first modified using suitable organic groups, and then the surface-modified particles are dispersed, using reactive siloxanes, where the siloxanes, too, contain organic groups.

Accordingly, the present invention provides a process for the preparation of a dispersion of inorganic particles in a liquid medium, in which inorganic particles which have been surface-modified so that they have at least one organic group on the surface are mixed in a liquid medium with a reactive organosiloxane.

Surprisingly, this method gave highly stable dispersions even when the filler level was relatively high. Even when the medium used was of relatively high viscosity, the dispersions obtained were easy to handle. The details of the invention are described below.

### DETAILED DESCRIPTION

The inorganic particles intended to be dispersed in the liquid medium can involve any of the inorganic particles known in the art. They can in particular involve inorganic particles usually used in products or compositions, e.g. as fillers, matrix-formers, pigments or for provision of other functional properties. The products or compositions can by way of example involve lacquers, moulding compositions, e.g. for plastics layers or for ceramics layers or for plastic mouldings or for ceramics mouldings. The dispersion prepared by the process of the invention is particularly suitable for resins, for example those used for the production of mouldings, where these have to achieve particularly high fill levels. Polymerization shrinkage can be reduced via a high proportion of fillers in polymerizable mixtures, e.g. in dental composites. The process is moreover particularly suitable for the dispersion of pigments in organic solvents. Dispersions of such pigments are used as additive or component in mouldings, in functional layers or in coatings. It is thus possible to control the flow properties of liquid media. The corresponding pigments can moreover improve specific properties of the materials or provide specific properties to the same, examples being hardness, colour, UV absorption, IR absorption, UV reflection or IR reflection, or semiconducting properties, high-refractive-index or low-refractive-index properties, microbicidal properties, conductive properties, antistatic properties, antislip properties, antiblocking properties, or adhesive properties. It is possible to improve feel and appearance, e.g. via matting. Also catalytic effects, e.g. photo-catalytic functions.

The inorganic particles can be composed of any desired suitable material. It is also possible to use a mixture of particles. Examples of inorganic particles are particles composed of an element, of an alloy, or of an elemental compound. The inorganic particles are preferably composed of compounds of metals or of semimetals, e.g. Si or Ge, or boron, particularly preferably of boron oxides, of metal oxides or of semimetal oxides, and this is intended here also to include hydrated oxides, oxide hydroxides or hydroxides.

Examples of metal compounds and compounds of semiconductor elements or boron are if appropriate hydrated oxides, such as ZnO, CdO, SiO₂ (in all modifications, e.g. precipitated or fumed silicas), GeO₂, TiO₂, ZrO₂, CeO₂, SnO₂, Al₂O₃ (in all modifications, in particular as corundum, boehmite, AlO(OH), also in the form of aluminium hydroxide), In₂O₃, La₂O₃, Fe₂O₃, Fe₃O₄, Cu₂O, Ta₂O₅, Nb₂O₅, V₂O₅, MoO₃ or WO₃, mixed oxides of boron, of metals and/or of semimetals, e.g. indium tin oxide (ITO), antimony tin oxide (ATO), fluorine-doped tin oxide (FTO) and mixed oxides with perovskite structure, e.g. BaTiO₃ and PbTiO₃, and also carbonates, sulphates, phosphates, silicates, zirconates, aluminates and stannates of elements, in particular of metals or Si, e.g. carbonates of calcium and/or magnesium, silicates, such as alkali metal silicates, talc, clays (kaolin) or mica, and sulphates of barium or calcium. Further examples of advantageous particles are magnetite, maghemite, spinels (e.g. MgO.Al₂O₃), mullite, escolaite, tialite, SiO₂.TiO₂, or bioceramics, e.g. calcium phosphate and hydroxyapatite. Core-shell particles are also suitable, e.g. those composed of a silica shell and of a core composed of metal oxide, i.e. metal oxide particles with a surface coating composed of SiO₂.

Particles composed of glass, of glass ceramic, or of ceramic, or of a material used for production of these, can be involved. Examples of glass are borosilicate glass, soda lime glass or quartz glass. Glass ceramics or ceramic can by way of example be based on the oxides SiO₂, BeO, Al₂O₃, ZrO₂ or MgO. Particles serving as fillers or as pigments can also be involved. Examples of industrially important fillers are fillers based on SiO₂, such as quartz, cristobalite, tripolite, novaculite, kieselgur, siliceous earth, fumed silicas, precipitated silicas and silica gels, silicates, such as talc, pyrophyllite, kaolin, mica, muscovite, phlogopite, vermiculite, wollastonite and perlites, carbonates, such as calcites, dolomites, chalk and synthetic calcium carbonates, carbon black, and sulphates, such as barium sulphate and calcium sulphate, iron mica, glasses, aluminium hydroxides, aluminium oxides and titanium dioxide, and zeolites.

Inorganic particles whose use is preferred are boron oxides, metal oxides or semimetal oxides, inclusive of hydrated oxides, oxide hydroxides or hydroxides, in particular SiO₂, in particular fumed silica, TiO₂, ZrO₂, Al₂O₃, in particular boehmite, glasses, iron oxides, ZnO and mixed oxides. It is particularly preferable to use fumed silica.

The particles that can be used are generally available commercially. Examples of SiO₂ particles are commercially available silica products, e.g. silica sols, e.g. Levasil® products, organosols from Nissan Chemicals, e.g. MA-ST, IPA-ST, or fumed silicas, e.g. the Aerosil® products from Degussa, e.g. Aerosil OX50, Aerosil 200, Aerosil 300, the HDK products from Wacker, and also the Cab-O-Sil products from Cabot.

Examples of aluminium oxide particles are commercially available products such as the Disperal products, and also Dispal products from Sasol, and also the aluminium oxides from the Aerosil process.

Examples of titanium dioxide particles are commercially available products such as P25 and P90 from Degussa, and also Hombitec and Hombicat from Sachtleben.

The particles used as fillers can usually be available commercially or prepared by conventional processes. The particles used can by way of example involve nanoparticles or microparticles. The specific surface area of the non-surface-modified inorganic particles is preferably greater than 50 m²/cm³, measured by the BET method using nitrogen.

The inorganic particles are, or have been, surface-modified to bear at least one organic group on the surface. The modification of the particle surface is familiar to the person skilled in the art and is often carried out in the prior art. The modification can use conventional processes. If the modification is carried out in a solvent, the modified inorganic particles can be isolated, but it is also possible to use the resultant dispersion without isolation in the present invention.

The surface modification using surface modifiers can improve the dispersibility of inorganic powders. Particularly in the modification of the particles using silanes, this is attributed to the reaction of the modifiers with reactive groups on the surface of the particles, e.g. hydroxy groups, which are in particular present in the case of oxide particles. To modify powders, it is theoretically sufficient that there is a monomolecular layer of modifiers, such as silane, on the surface. In practice, concentrations of about 1% are recommended for the modification of inorganic powders; e.g. in the Degussa brochure “Sivento Silanes for Treatment of Fillers and Pigments”, p. 10.

Surface-modified particles of this type are commercially available, examples being hydrophobized powders, such as hydrophobized silicas, e.g. Aerosil® R 9200 and Aerosil® R 7200 from Degussa, fine-particle silicas, marketed by Wacker with trade name HDK, VP AdNano® Z 805 hydrophobized zinc oxide from Degussa, hydrophobic titanium dioxide, e.g. Hombitan® R320 from Sachtleben. These types of commercially available powders are also, of course, suitable as surface-modified component in the process of the invention. Commercially available dispersions of modified particles are moreover suitable as surface-modified components in the process of the invention. Examples of these dispersions are modified silica sols from Clariant (e.g. Highlink NanOG grades), and the modified silica sols from Nissan Chemicals, e.g. MEK-ST, MEK-ST-MS.

If non-modified particles are starting materials, the first step modifies the surface, giving particles having organic groups on the surface. The processes for the preparation of the modified particles are familiar to the person skilled in the art. In particular, the inorganic particles can be reacted with at least one surface modifier which has at least one functional group that interacts with surface groups on the inorganic particles and which has at least one organic group. In one variant, the preparation of the inorganic particles can be take place in the presence of the surface modifiers, so that the modification takes place in situ during the preparation process. It is also possible to use colloidal dispersions of modified particles, prepared by way of example according to DE A 10304849.

The reaction takes place under conditions such that binding of the modifier takes place on the surface of the particles, e.g. via chemical bonding or interaction. The conditions are naturally dependent on the nature of the particles and of the surface modifiers. Simple stirring at room temperature can be sufficient, but energy input, e.g. via heating, or high shear (chemo-mechanical reaction), and/or catalysis, e.g. using acids or bases, can also sometimes be necessary. The degree of covering of the particle surfaces by the modifiers can by way of example be controlled via the quantitative proportion used of the starting materials.

The person skilled in the art is aware that there are generally groups present on the surface of particles, and that these surface groups can be functional groups, which are generally relatively reactive. By way of example, there are residual valences are present on the surface of particles, examples being hydroxy groups and oxy groups, e.g. in the case of metal oxide particles. The surface modifier has firstly at least one functional group, which can interact or react chemically with reactive groups present on the surface of the particles, to give binding. The binding can take place via chemical bonding, such as covalent bonding, inclusive of coordinative bonding (complexes), or ionic (salt-type) bonds of the functional group to the surface groups of the particles, and interactions that may be mentioned here by way of example are dipole-dipole interactions, polar interactions, hydrogen bonding and van der Waals interactions. The formation of a chemical bond is preferred. By way of example, therefore, an acid/base reaction, complexing, or esterification can take place between the functional groups of the modifier and the particle. Surface modifiers of this type are known to the person skilled in the art, and that person can readily select those suitable for the respective particles.

The functional group comprised by the surface modifier is, for example, carboxylic acid groups, acyl chloride groups, ester groups, nitrile groups and isonitrile groups, OH groups, alkyl halide groups, SH groups, epoxide groups, anhydride groups, amide groups, primary, secondary and tertiary amino groups, Si—OH groups or hydrolysable moieties of silanes (groups Si—X explained below), or acidic C—H groups, examples being β-dicarbonyl compounds, and also organic derivatives of inorganic acids.

The modifier can also comprise more than one such functional group, as for example is the case in amino acids or EDTA.

Examples of suitable modifiers are mono- and polycarboxylic acids, corresponding anhydrides, acyl chlorides, esters and amides, alcohols, alkyl halides, amino acids, imines, nitriles, isonitriles, epoxy compounds, mono- and polyamines, β-dicarbonyl compounds, silanes and metal compounds, where these have a functional group that can react with the surface groups of the particles, and in each case also have an organic group, and also esters of inorganic acids, e.g. mono-, di- or triesters of ortho-, oligo-, or polyphosphoric acid, esters of sulphuric acid, and also esters of sulphonic acid. The reagent preferably used for surface modification depends substantially on the nature of the powder to be modified. For modification of SiO₂, it is particularly preferable to use silanes. It is generally possible to use one or more modifiers.

The surface modifier also comprises the organic group with which the particles are modified. The organic group can, for example, be an organic group having one or more functional groups, or an organic hydrophobic and/or oleophobic group. Examples of these organic groups are alkyl groups, alkenyl groups, such as vinyl groups or allyl groups, alkynyl groups, or aryl groups, inclusive of the corresponding cyclic groups, such as cycloalkyl, and in each case these can bear one or more, preferably one, functional group. The alkyl groups, alkenyl groups and alkynyl groups can have interruption by oxygen groups or by —NH groups. The organic group can by way of example contain from 1 to 18 carbon atoms, ignoring any carbon atoms present in any functional group present.

Examples of suitable functional groups are epoxy groups, hydroxy groups, ether groups, amino groups, monoalkylamino group, dialkylamino groups, unsubstituted or substituted anilino groups, amide groups, carboxy groups, acrylic groups, acryloxy groups, methacrylic groups, methacryloxy groups, silyl groups, mercapto groups, cyano groups, alkoxy groups, isocyanato groups, aldehyde groups, alkylcarbonyl groups, anhydride groups and phosphoric acid groups. It is preferable to use modifiers which contain an organic group which has a functional group, in particular which has a methacrylic function. Examples of specific organic groups are mentioned below for the silanes of the formula (I) particularly preferred for the modification of SiO₂. The same organic groups can also be applied by way of other abovementioned modifiers to the surface of the inorganic particles. The organic group is generally the modifier without the functional group that interacts with the reactive groups of the particle surface. By way of example, in the case of a carboxylic acid, the organic group is the moiety remaining in the absence of the carboxy group. The organic group can moreover if appropriate have conventional substituents, such as chlorine or fluorine.

Preferred surface modifiers are hydrolysable silanes having at least one non-hydrolysable organic group, as defined above. These are therefore explained in more detail. Corresponding information is also applicable analogously to other modifiers, in particular in relation to suitable organic groups. Suitable hydrolysable silanes having a non-hydrolysable organic group have by way of example the general formula

RₐSiX₍₄₋ₐ₎ (I)

in which R is identical or different and is a non-hydrolysable organic moiety which if appropriate has one or more functional groups, X is a hydrolysable group or OH, and a is 1, 2 or 3, preferably 1.

Examples of the hydrolysable group X are hydrogen or halogen (F, Cl, Br or I), alkoxy (preferably C₁₋₆-alkoxy, e.g. methoxy, ethoxy, n-propoxy, isopropoxy and butoxy), carboxy, amino, monoalkylamino or dialkylamino preferably having from 1 to 12, in particular from 1 to 6, carbon atoms in the alkyl group(s), aryloxy (preferably C₆₋₁₀-aryloxy, e.g. phenoxy), acyloxy (preferably C₁₋₆-acyloxy, e.g. acetoxy or propionyloxy) or alkylcarbonyl (preferably C₂₋₇-alkylcarbonyl, e.g. acetyl).

Examples of the non-hydrolysable organic moiety R are alkyl (preferably C₁₋₃₀-alkyl, such as methyl, ethyl, n-propyl, iso-propyl, n-butyl, sec-butyl and tert-butyl, pentyl, hexyl or cyclo-hexyl), alkenyl (preferably C₂₋₆-alkenyl, e.g. vinyl, 1-propenyl, 2-propenyl and butenyl), alkynyl (preferably C₂₋₆-alkynyl, e.g. acetylenyl and propargyl) and aryl (preferably C₆₋₁₀-aryl, e.g. phenyl and naphthyl).

The non-hydrolysable organic moiety R having a functional group can comprise, as functional group, by way of example, an epoxy group (e.g. glycidyl group or glycidyloxy group), hydroxy group, ether group, amino group, monoalkylamino group, dialkylamino group, unsubstituted or substituted anilino group, amide group, carboxy group, acrylic group, acryloxy group, methacrylic group, methacryloxy group, mercapto group, cyano group, alkoxy group, isocyanato group, aldehyde group, alkylcarbonyl group, anhydride group and phosphoric acid group. These functional groups have bonding to the silicon atom by way of alkylene groups, alkenylene groups or arylene groups, which may have interruption by oxygen groups or by —NH groups. The bridging groups preferably contain from 1 to 18 carbon atoms, preferably from 1 to 8 and in particular from 1 to 6. The divalent bridging groups mentioned and any substituents present derive, by way of example, as is the case with the alkylamino groups, from the abovementioned organic groups R without functional groups, i.e. from the alkyl moieties, alkenyl moieties, aryl moieties, alkaryl moieties or aralkyl moieties. The moiety R can also have more than one functional group.

Preferred organic groups without functional groups are alkyl groups, as defined above. Examples of hydrolysable silanes of this type are methyltriethoxysilane, propyltrimethoxysilane, hexadecyltrimethoxysilane, dodecyltriethoxysilane. It is also possible to use if appropriate fluorinated alkyl groups as organic groups. Alkyl groups and fluorinated alkyl groups are suitable by way of example as hydrophobic and/or oleophobic groups.

Examples of non-hydrolysable moieties R having functional groups are glycidyloxyethyl, glycidyloxypropyl, aminopropyl, (meth)acryloxymethyl, (meth)acryloxyethyl, (meth)acryloxypropyl and 3-hydroxypropyl. Particular preference is given to methacryloxyalkyl, in particular methacryloxypropyl. (Meth)acrylic means methacrylic or acrylic. Specific examples of corresponding silanes are glycidyloxypropyltrimethoxysilane, 3-aminopropyltrimethoxysilane, 3-aminopropyltriethoxysilane, hydroxymethyltriethoxysilane, 3-(meth)acryloxypropyltriethoxysilane, 3-(meth)acryloxypropyltrimethoxysilane, 3-(meth)acryloxymethyltrimethoxysilane and 3-(meth)acryloxymethyltriethoxysilane.

Specific examples of other surface modifiers that can be used for the introduction of organic groups are saturated or unsaturated mono- and polycarboxylic acids, e.g. acrylic acid, methacrylic acid or crotonic acid, mono- and polyamines, such as methylamine, or ethylenediamine, β-dicarbonyl compounds, such as acetylacetone, or amino acids, organic derivatives of sulphuric acid, such as alkyl sulphates or fatty alcohol sulphates, esters of sulphonic acids, such as alkyl sulphonic acids and alkyl sulphonates, organic phosphates, such as (alkyl)ethoxylated phosphoric acids or lecithin, polyacids, such as polyhydroxyaspartic acid and polyhydroxystearic acid. Other examples are 1H,1H-penta-decafluorooctanol, octanol, stearic acid, oleic acid, hexanolyl chloride, methyl hexanoate, hexyl chloride and nonafluorobutyl chloride.

The molecular weight of the surface modifier is preferably not more than 10 000 and more preferably not more than 5 000, but it is also possible to use modifiers having higher molecular weight.

After the surface modification of the inorganic particles they are dispersed, in the second step, with use of specific siloxanes, into the liquid medium or, respectively, the components forming the matrix. As has been said, it is naturally also possible to use commercially available surface-modified inorganic particles directly to the second step. The siloxanes used involve organosiloxanes, i.e. siloxanes which have at least one organic group. In one specific embodiment of the process of the invention, the, or an, organic group of the organosiloxane corresponds to the, or an, organic group located on the surface-modified inorganic particles used.

Examples of suitable mutually corresponding organic groups are alkyl groups, epoxide groups, hydroxy groups, ether groups, amino groups, monoalkylamino groups, dialkylamino groups, unsubstituted or substituted anilino groups, amide groups, carboxy groups, acrylic groups, acryloxy groups, methacrylic groups, methacryloxy groups, mercapto groups, cyano groups, alkoxy groups, isocyanato groups, aldehyde groups, alkylcarbonyl groups, anhydride groups and phosphoric acid groups-, carboxylic acid groups, ester groups, imine groups and imide groups.

There can be any desired spacers separating the organic group from the silicon atom of the siloxane. Equally, there can be any desired spacers separating the same organic group from the particle surface. The respective spacers do not have to be identical and they can also bear one or more functional groups. It is preferable that the organic group of the organosiloxane comprises a methacrylate function.

By way of example, the siloxanes can be obtained via reaction of at least one hydrolysable silane having at least one non-hydrolysable organic group with water. The reaction with water hydrolyses the hydrolysable silanes to form the hydrolysates and generally causes at least some degree of condensation. The sol-gel process is particularly suitable for this purpose. The reaction can, if appropriate, take place in the presence of catalysts. The period between preparation of the organosiloxane and its use is preferably not longer than three months, particularly preferably not longer than one month. The siloxanes prepared therefore retain reactivity such that a further reaction can take place by way of Si—O groups, i.e. saturation of the Si—O groups is not yet complete. In general terms, reactive organosiloxanes are those organosiloxanes which retain hydrolysable groups on Si atoms, in particular groups X as defined in formula (I), and/or have hydrolysed groups (OH).

The organosiloxane is in particular a condensate of one or more silanes, where at least one silane has the formula (I) RₐSiX₍₄₋ₐ₎ as defined above for the surface modifiers, where a=1 (RSiX₃). Preparation of the organosiloxane in particular uses at least 10 mol %, preferably at least 50 mol %, more preferably at least 80 mol % or from 80 to 100 mol %, of one or more silanes of the formula (I), where a=1, based on all of the silanes used for the condensate. In one preferred embodiment, all of the silanes from which the organosiloxane or condensate is formed are silanes of the formula (I), where a=1. These silanes of the formula (I), where a=1 have 3 reactive silanol functions after hydrolysis of the groups X. The condensation of 2 silanol groups per molecule intrinsically leads to formation of a (linear) skeletal structure. The organosiloxane therefore contains further Si—O functionalities in the molecular skeleton, in addition to terminal reactive Si—O groups.

With no intention to become bound to any theory, it is assumed that the siloxanes in the liquid medium form a structure in which the surface-modified particles are particularly advantageously incorporated. By virtue of the use of reactive siloxanes, the dispersion procedure takes place in the presence of chemically reactive organosiloxanes. By virtue of the prior surface modification of the powders, however, a chemical reaction of the reactive siloxane by way of Si—O functions with the powder surface is inhibited. The compatibility of the components to one another can be adjusted appropriately via the organic groups of the modified particles, and also of the organosiloxanes. In the embodiment which uses chemically identical groups in siloxane and modified particles, appropriate adjustment of the components in the mixture is particularly simple.

The use of trifunctional silanes of the formula I (a=1) leads to formation of branched siloxane structures in the condensation reaction. In the liquid medium, these can form flat (2-dimensional) and 3-dimensional networks. The content of tri-functional silanes in the reaction mixture is therefore used not only to control the number of reactive Si—O functions but also to control the density of the resultant structure. The multidimensional siloxane network infiltrates the liquid medium and bears organic groups which are advantageous for the dispersion of the surface-modified particles, permitting embedding of the particles into the network structure.

The desired organosiloxane can be obtained via suitable adjustment of the parameters, e.g. selection of the starting silanes, degree of condensation, solvent, temperature, water concentration, catalyst, duration or pH. The person skilled in the art is aware of the processes for the preparation of these organosiloxanes. Details of the sol-gel process are found by way of example in C. J. Brinker, G. W. Scherer: “Sol-Gel Science—The Physics and Chemistry of Sol-Gel-Processing”, Academic Press, Boston, San Diego, New York, Sydney (1990).

The hydrolysable silanes used having at least one non-hydrolysable organic group, used for the preparation of the organosiloxanes, preferably comprise the silanes defined above of the formula (I) or a mixture thereof. If appropriate, it is also possible, in addition, to use hydrolysable silanes without any non-hydrolysable group, e.g. compounds of the formula SiX₄, in which X is defined as in formula (I), these then likewise being incorporated into the organosiloxane. However, it is preferable to use organosilanes of the formula (I) for the preparation of the organosiloxanes, and it is particularly preferable to use only one silane of the formula (I).

It is preferable to use hydrolysable silanes of the formula (I) in which X is alkoxy, carboxy, amino or halogen. At least one non-hydrolysable moiety of the silane (the organic group R in the formula (I)) used for the preparation of the siloxane is functionally identical with the organic group on the surface of the modified particles. The organosiloxane preferably has an organic group having a methacrylic function. Preferred silanes for the preparation of the organosiloxanes are accordingly silanes of the formula (I) in which R is an organic group having a methacrylic function, particular preference being given here to the use of γ-methacryloxypropylsilane.

It can, of course, be advantageous to use in each case the same silane of the formula (I) for the surface modification and for the preparation of the organosiloxane. However, it is also possible to use different silanes, or else different surface modifiers, as long as the organosiloxane and the inorganic particles have organic groups which are functionally identical.

As explained above, the reaction of silanes with water for the preparation of siloxanes is known per se, and the person skilled in the art can readily select the respective parameters on the basis of the starting substances used and of the desired properties. Examples of catalysts suitable for the reaction are acids, bases and fluoride ions. The reaction can be carried out with or without solvent. Examples of suitable solvents are water and organic solvents, e.g. alcohols, ketones or esters, or a mixture thereof. With respect to specific examples of organic solvents that can be used, reference is made to the corresponding examples given below for the liquid media. The reaction of the silanes can take place separately or in the presence of the surface-modified particles. The temperature and the time for the reaction can be selected within a wide range and also, of course, depends by way of example on the hydrolysis resistance of the silanes used, on the nature and amount of the catalyst used, etc. The reaction is generally carried out at least as far as the clear point. The reaction can generally be carried out, for example, at a temperature in the range from 15 to 150° C., for example over a period of from 15 to 360 min.

Volatile components can then, if necessary, be removed completely or to some extent by distillation. However, the mixture obtained can also be used without distillation. Distillation to remove components can by way of example be useful in order to achieve a further increase in the degree of reaction, i.e. the degree of condensation of the organosiloxanes, by shifting the equilibrium, or in order to remove undesired by-products, e.g. methanol, which forms during hydrolysis of methoxysilanes.

The modified particles are mixed in a liquid medium with the organosiloxane, in order to obtain the dispersion. The liquid medium can involve any desired liquid medium, and in particular involves a solvent, such as water or an organic solvent, a binder component, or a mixture thereof. The liquid medium used can also, if appropriate, comprise a non-liquid or highly viscous binder component, via mixing with a suitable solvent. However, the binder component preferably involves a liquid binder component. Particularly if binder components are used, the liquid medium can be a medium with a certain viscosity. Surprisingly, the process of the invention delivers good results even when the viscosity of the liquid medium (starting medium) used, i.e. without addition of the other components, is high, an example of a viscosity η being >100 mPa s (dynamic viscosity, measured at 23° C. using parallel-plate geometry with gap width of 0.25 mm). The viscosity of the starting medium is advantageously at most 42 Pa s (dynamic viscosity, measured at 23° C. using parallel-plate geometry with gap width of 0.25 mm). In one preferred embodiment, the liquid medium comprises, or is, a liquid binder component, in particular a reactive resin, and particularly preferably an acrylate resin.

The binder component can by way of example involve one or more monomers, oligomers, polymers or reactive resins. These binder components are by way of example generally used as matrix-forming component. These binder components are generally reactive and are converted via polymerization or curing by way of example into the solid plastics products or solid synthetic resins products. A wide variety of the same is commercially available.

In another preferred embodiment, the liquid medium comprises or is a solvent, in particular an organic solvent.

Examples of solvents suitable for the liquid medium are water and organic solvents, such as alcohols, e.g. methanol, ethanol and 1-propanol, esters, e.g. butyl acetate and ethyl acetate, mono-, di- and triglycerides, e.g. fatty acid esters, e.g. palmitic acid esters and coconut acid esters, ketones, e.g. acetone, ethyl methyl ketone and methyl isobutyl ketone, cyclohexanone, silicone oils, e.g. cyclomethicone and dimethicone, aliphatic and aromatic hydrocarbons, e.g. pentane, heptane, isooctane, cyclohexane, toluene and xylene, and ethers, e.g. diethyl ether, polyethylene glycols and their derivatives.

Examples of Liquid Binder Components are:

Acrylates and Acrylate Resins:

(Meth)acrylic acid, esters of (meth)acrylic acid with mono-, di- and polyalcohols, e.g. hexamethylenediol diacrylate, trigema, PETA, Di-PETA, Bis-GMA, TEGDMA, phosphonic acid acrylates, hydroxyethyl methacrylate, glycerol 1,3-dimethacrylate, and acrylate-modified oligomers and polymers; preparations of acrylate resins are commercially available, e.g. with trade mark Laromer® from BASF, examples of grades being LR 8765, LR 8863, LR8800;

Epoxies and Epoxy Resins:

Glycidic ethers, such as bisphenol A glycidic ether and its derivatives, commercially available epoxy resins, e.g. Epikote® 1100, Epikote® 815, Epikote® 235 from Hanf and Nelles chemische Produkte; and also alkyd resins, silicone resins, poly-hydroxy compounds, such as glycerol, polyether polyols and poly-ester polyols, mono- and diolefins, such as pentene and terpinol.

There is no restriction on the sequence in which the three components, i.e. the surface-modified particles, the organosiloxane and the liquid medium, are mixed with one another in order to obtain a dispersion. The surface-modified particles can, for example, be incorporated as dry powder or dispersion into the liquid medium, where the organosiloxane is already present in the liquid medium, or is added simultaneously or is not added until subsequently. The surface-modified particles can also by way of example be mixed as dry powder or dispersion with the organosiloxane, and this mixture can then be added to the liquid medium. The organosiloxane can also be used as it stands or in a solvent, e.g. in the form of a sol. The organosiloxane can therefore be prepared separately or in the presence of the surface-modified particles. Other variants are also conceivable, for example where only a portion of a component is first added and the remainder is added at a later juncture.

The mixing or incorporation of the components to achieve a dispersion can take place using any desired mixing apparatus, e.g. using a dispersing machine. Examples of suitable dispersing machines are jet-stream mixers, dissolvers, nozzle jet dispersers, homogenisers, turbo mixers, mills, such as mills using free-running grinder devices, e.g. stirred bead mills, mortar mills, colloid mills, kneaders, such as shear-roll kneaders, and roll mills.

The amounts and proportions of the components for the dispersion can be selected from a wide range. By way of example, it is possible to use from 1 to 90% by weight of siloxane in the mixture, preferably from 5 to 50% by weight and particularly preferably from 5 to 30% by weight, based on the entire composition. The amount of surface-modified inorganic particles is preferably selected in such a way that the concentration of the particles in the dispersion is greater than 2% by volume, preferably greater than 3% by volume.

Other additives can be present in the dispersion as a function of the intended application, examples being colorants, hardeners, crosslinking agents, and flow-control agents, which can be added after preparation of the dispersion or, if appropriate, beforehand.

Very surprisingly, it is possible to achieve highly stable dispersions of the inorganic particles, even in relatively viscous liquid media. Relatively high filler levels can moreover be achieved. The dispersions are suitable, for example, for lacquers or moulding compositions, which after curing can be converted into coatings or into mouldings. The dispersions in reactive resins are particularly suitable for dental composites. Other application sectors for the dispersions of the invention are additives in coating materials, e.g. scratch resistance additives or UV-protection additives.

### EXAMPLES

### Experiment 1, Preparation of a Surface-Modified Fumed SiO₂

Aerosil A 200 (Degussa, 200 g) is introduced into butanone (Sigma-Aldrich, 800 g), with stirring. Methacryloxypropyltrimethoxysilane (ABCR, 36.22 g), and also acetic acid (Sigma-Aldrich, 99-100%, 2.01 g) are added to the mixture and it is stirred for one hour at room temperature. The dispersion is then concentrated at reduced pressure using a bath temperature of 65° C. in a rotary evaporator. The powder, still wet, is transferred to a vacuum drying oven, where it is dried for 14 h at 80° C. The surface-modified powder is characterized by thermogravimetric methods (TG). Weight loss is 0.8% by weight up to a temperature of 244° C. Weight loss of 6.4% by weight occurs between 260 and 360° C. At 800° C., the weight is 90.05% of the surface-modified Aerosil.

### Experiment 2

Preparation of the organosiloxane: H₂O (21.95 g) is admixed with methacryloxypropyltrimethoxysilane (ABCR, 201.6 g), with stirring. Acetic acid (99-100%, 2.93 g) is added to this mixture. The mixture is stirred at room temperature for 30 min, until a clear, single-phase solution is obtained.

Preparation of the dispersion: the siloxane from a is admixed with Laromer 8863 (BASF, 800 g). Aerosil R7200 (Degussa, 576 g) is then introduced in portions. The mixture is milled by passing through a stirred bead mill (PML1, Bühler AG). Yttrium-stabilized zirconium oxide beads (2.52 kg) with diameter of 1.75 mm are used as bead fill, giving 70% filling of the grinder vessel. The rotation rate during milling is 1200 rpm. A total of 10 passes is used to mill the mixture.

The dispersion has a shelf life of 4 months at room temperature. It exhibits shear-thinning behaviour. Viscosity at a shear rate of 1 s⁻¹ is 15.2 Pa s and at a shear rate of 150 s⁻¹ is 3.01 Pa s.

### Experiment 3

Preparation of the organosiloxane: H₂O (0.88 g) is admixed with methacryloxypropyltrimethoxysilane (ABCR, 8 g), with stirring. Acetic acid (99-100%, 0.32 g) is added to this mixture. The mixture is stirred at room temperature for 30 min, until a clear, single-phase solution is obtained.

Preparation of the dispersion: the siloxane from a is admixed with Laromer 8800 (150.3 g). Aerosil R711 (40 g) is then incorporated. A roll mill is used to shear the resultant mixture, for which purpose it is subjected to 12 passes.

The dispersion exhibits shear-thinning behaviour. Viscosity at a shear rate of 100 s⁻¹ is 52.2 Pa s and at a shear rate of 200 s⁻¹ is 31 Pa s.

### Experiment 4 (Comparative Example)

Aerosil R711 is added in portions to the Laromer 8800. It is impossible to incorporate more than 20 g.

### Experiment 5

Preparation of hydrolysate: H₂O (0.87 g) is admixed with methacryloxypropyltrimethoxysilane (8.0 g, ABCR), with stirring. Acetic acid (99-100%, 0.32 g) is added to this mixture. The mixture is stirred at room temperature for 30 min, until a clear, single-phase solution is obtained.

Preparation of the dispersion: the organosiloxane from a is admixed with Laromer 8800 (150 g). Aerosil R7200 (40 g) is then incorporated. A roll mill is used to shear the resultant mixture, for which purpose it is subjected to 12 passes.

The dispersion exhibits shear-thinning behaviour. Viscosity at a shear rate of 101 s⁻¹ is 31.5 Pa s and at a shear rate of 200 s⁻¹ is 27.4 Pa s.

1. Process for the preparation of a dispersion of inorganic particles in a liquid medium, in which inorganic particles which have been surface-modified so that they have at least one organic group on the surface are mixed in a liquid medium with an organosiloxane.

2. Process according to claim 1, where the organosiloxane is a condensate of one or more silanes comprising a silane of the formula RSiX₃ (I), in which R is a non-hydrolysable organic moiety and X is a hydrolysable group or OH.

3. Process according to claim 2, wherein the non-hydrolysable organic moiety has one or more functional groups.

4. Process according to claim 2, where at least 10 mol % of the silanes for the condensate are a silane of the formula RSiX₃ (I).

5. Process according to claim 2, where at least 50 mol % of the silanes for the condensate are a silane of the formula RSiX₃ (I).

6. Process according to claim 2, where at least 80-100 mol % of the silanes for the condensate are a silane of the formula RSiX₃ (I).

7. Process according to claim 1, where the organosiloxane is a reactive organosiloxane.

8. Process according to claim 1, characterized in that the organosiloxane is prepared via reaction of at least one hydrolysable silane having at least one non-hydrolysable organic group with water.

9. Process according to claim 1, characterized in that the liquid medium, the surface-modified inorganic particles and the organosiloxane are mixed in a dispersing machine.

10. Process according to claim 1, characterized in that the concentration of the surface-modified inorganic particles in the dispersion is greater than 3% by volume.

11. Process according to claim 1, characterized in that the specific surface area of the non-surface-modified inorganic particles is greater than 50 m²/cm³ (measured by the BET method using nitrogen).

12. Process according to claim 1, characterized in that the inorganic particles used comprise SiO₂.

13. Process according to claim 12, characterized in that the inorganic particles used comprise fumed silica.

14. Process according to claim 1, characterized in that the liquid medium is water, an organic solvent, a binder component or a mixture thereof.

15. Process according to claim 13, characterized in that the liquid medium comprises, as binder component, or is an organic resin.

16. Process according to claim 13, characterized in that the liquid medium comprises or is an organic solvent.

17. Process according to claim 1, where at least one organic group of the organosiloxane corresponds to the at least one organic group on the surface of the inorganic particles.

18. Process according to claim 1, characterized in that the viscosity η of the liquid medium is >100 mPa s (dynamic viscosity, measured at 23 C using parallel-plate geometry).

19. Process according to claim 1, characterized in that the liquid medium comprises or is a reactive resin.

20. Process according to claim 1, characterized in that the liquid medium comprises or is an acrylic resin.

21. Process according to claim 1, characterized in that the organosiloxane has been formed from structural units of only one organosilane.

22. Process according to claim 1, characterized in that the at least one organic group of the organosiloxane comprises a methacrylic function.

23. Process according to claim 1, characterized in that a γ-methacryloxypropylsilane is used for the surface modification of the inorganic particles and/or for the preparation of the organosiloxane.

24. Process according to claim 1, characterized in that the dispersion prepared is a dental composite composition, a lacquer or a moulding composition.

25. Dispersion of inorganic particles in a liquid medium, comprising surface-modified inorganic particles having at least one organic group on the surface, a liquid medium and an organosiloxane having an organic group which corresponds to the organic group on the surface of the inorganic particles, obtainable by the process of claim 1.

26. Use of dispersions obtainable according to claim 24 as additive or components in a lacquer, coating, moulding composition, or dental material.
